(19) United States
(12) Patent Application Publication
(10) Pub. No.: US 2018/0091875 A1
(43) Pub. Date: Mar. 29, 2018

(54) MONITORING THE HEALTH AND SAFETY OF PERSONNEL USING UNMANNED AERIAL VEHICLES

(71) Applicant: International Business Machines Corporation, Armonk, NY (US)

(72) Inventors: Donald L. Bryson, Chattanooga, TN (US); Eric V. Kline, Rochester, MN (US); Sarbajit K. Rakshit, Kolkata (IN)

(21) Appl. No.: 15/278,147

(22) Filed: Sep. 28, 2016

(51) Int. Cl. H04Q 9/00; G05D 1/00; B64C 39/02

(52) U.S. Cl. CPC H04Q 9/00; G05D 1/0088; B64C 2201/12; H04Q 2209/40; B64C 39/024

(57) ABSTRACT

Aspects include a method, system and computer program product for monitoring a person for a condition. The method comprises defining a person to be monitored with an unmanned aerial vehicle (UAV), the UAV having at least one sensor. An area is defined associated with the person. A condition is defined associated with the person. The UAV is deployed to monitor the person. The condition is detected within the area. A signal is transmitted based on the detection of the condition.

### BACKGROUND

The present invention relates generally to a system and method for monitoring the health and safety of a person using an unmanned aerial vehicle and, more specifically, to a system and method for using unmanned aerial vehicles for monitoring a health or safety related condition of a person or persons.

Unmanned aerial vehicles (UAVs) can be used to achieve a certain set of needs or tasks such as using sensors such as a camera or an infrared sensor for monitoring an area.

### SUMMARY

Embodiments include a method, system and computer program product for monitoring a person for a condition. The method comprises defining a person to be monitored with an unmanned aerial vehicle (UAV), the UAV having at least one sensor. An area is defined associated with the person. A condition is defined associated with the person. The UAV is deployed to monitor the person. The condition is detected within the area. A signal is transmitted based on the detection of the condition.

Additional features are realized through the techniques of the present invention. Other embodiments and aspects of the invention are described in detail herein and are considered a part of the claimed invention. For a better understanding of the invention with the features, refer to the description and to the drawings.

### BRIEF DESCRIPTION OF THE DRAWINGS

The subject matter which is regarded as the invention is particularly pointed out and distinctly claimed in the claims at the conclusion of the specification. The forgoing and other features of embodiments of the invention are apparent from the following detailed description taken in conjunction with the accompanying drawings in which:

FIG. 1 depicts a block diagram of an unmanned aerial vehicle in accordance with an embodiment of this disclosure;

FIG. 2 depicts a block diagram of a controller for an unmanned aerial vehicle in accordance with an embodiment of this disclosure;

FIG. 3 depicts a plan view of a monitoring system in accordance with some embodiments of this disclosure;

FIG. 4 depicts a flow diagram of a method of monitoring subject personnel in accordance with some embodiments of this disclosure;

FIG. 5 depicts a plan view of a monitoring system for a plurality of personnel at an event in accordance with some embodiments of this disclosure;

FIG. 6 depicts a flow diagram of a method of monitoring the plurality of personnel of FIG. 5 in accordance with some embodiments of this disclosure;

FIG. 7 depicts a plan view of a monitoring system for external factors in accordance with some embodiments of the invention;

FIG. 8 depicts a cloud computing environment according to an embodiment of the present invention; and

FIG. 9 depicts abstraction model layers according to an embodiment of the present invention.

### DETAILED DESCRIPTION

Embodiments of the present disclosure are directed to a system and method for monitoring a person and detecting a condition. Embodiments of the present disclosure provide for monitoring a route and time period with an unmanned autonomous vehicle (“UAV”) for a pre-determined subject , e.g., person, to proceed from a starting location to an end location and transmitting a signal when the route or time period deviates from a predetermined threshold. Embodiments of the present invention further provide for monitoring a plurality of persons at an event with a UAV, such as athletes for example, and determining if an undesirable physical condition or potential physical condition has occurred. Still further embodiments, provide for monitoring an area, such as a beach, with a UAV to identify large animals and perform an action, where reasonable and appropriate, to protect a subject.

It should be appreciated that while embodiments herein describe monitoring the health and safety of a person, this is for exemplary purposes and the claimed invention should not be so limited. In other embodiments, the subject may be an animal such as cattle that are monitored by a farmer.

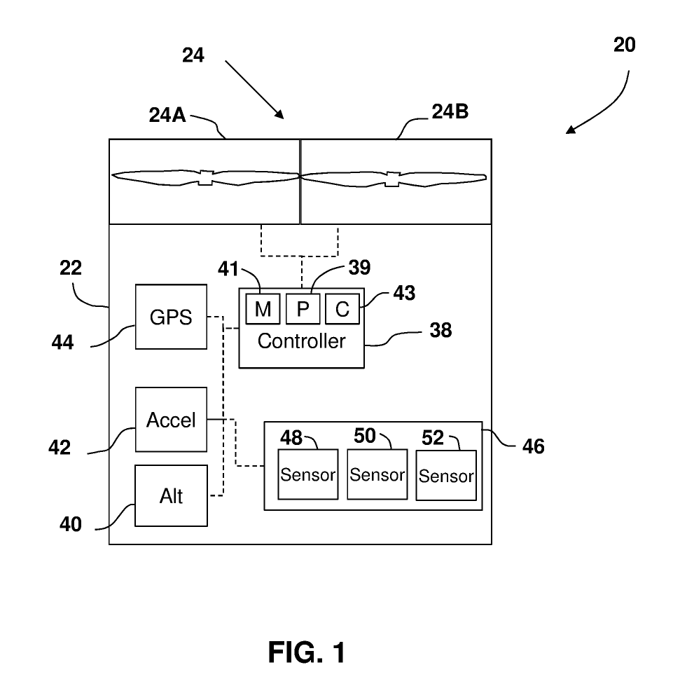

Referring now to FIG. 1, an embodiment is shown of a UAV 20. As used herein, the terms UAV or “drone” refer to an aerial vehicle capable of operating autonomously from a human operator to perform a predetermined function, such as perform monitoring functions. The UAV 20 includes a fuselage 22 that supports at least one thrust device 24. In an embodiment, the UAV 20 includes a plurality of thrust devices 24A, 24B, such as four thrust devices arranged about the periphery of the fuselage 22. In an embodiment, the thrust devices 24 include propeller member that rotates to produce thrust. The thrust devices 24 may be configurable to provide both lift (vertical thrust) and lateral thrust (horizontal thrust). The vertical and horizontal components of the thrust allow the changing of the altitude, lateral movement and orientation (attitude) of the UAV 20.

The UAV 20 includes a controller 38 having a processing circuit. The controller 38 may include processors that are responsive to operation control methods embodied in application code. These methods are embodied in computer instructions written to be executed by the processor, such as in the form of software. The controller 38 may further include additional circuits, such as but not limited to one or more processors 39, memory circuits 41 and communications circuits 43 for example. The communications circuit may be via a wireless communications medium. The wireless communications medium may include WiFi (e.g. IEEE 802.11), a Bluetooth® (e.g. IEEE 802.15.1 and its successors), RFID, near field communications (NFC), or cellular (e.g. LTE, GSM, EDGE, UMTS, HSPA and 3GPP cellular network technologies) for example.

The controller 38 is coupled to transmit and receive signals from the thrust devices 24 to determine and change their operational states (e.g. adjust lift from thrust devices 24, change the position of the UAV 20). The controller 38 may further be coupled to one or more sensor devices that enable to the controller to determine the position, orientation and altitude of the UAV 20. In an embodiment, these sensors may include an altimeter 40, a gyroscope or accelerometers 42 or a global positioning satellite (GPS) system 44. The controller 38 may use these input to operate the thrust devices 24 to move the UAV 20 to a predetermined position and orientation, and to maintain the UAV 20 in that position and orientation.

The UAV 20 may further include a personnel sensor 46 that is used for monitoring the subject personnel. The personnel sensor 46 may include a plurality of sensors, such as a camera 48, a thermal imaging sensor 50 and an acoustical sensor 52. The personnel sensors 46 may be used to determine when undesirable conditions associated with the subject personnel may occur or may have occurred. These sensors 46 may be used in cooperation with other systems, such as GPS system 44 to determine if the subject personnel deviates from a predetermined route or if the subject person is delayed along the predetermined route. As discussed in more detail herein, in other embodiments, the data acquired by the by the personnel sensors may be used to determine a physical condition, such as a player that may have received an impact that has a potential to cause a head injury or concussion for example.

In still further embodiments, the personnel sensor 46 may include a distance meter, a RADAR type device, a LIDAR type device, or a SONAR type device. As discussed in more detail herein, these types of sensors may allow for the direct non-contact measurement of the distance from the UAV 20 to the subject and allow for more precise determination of the subject's location.

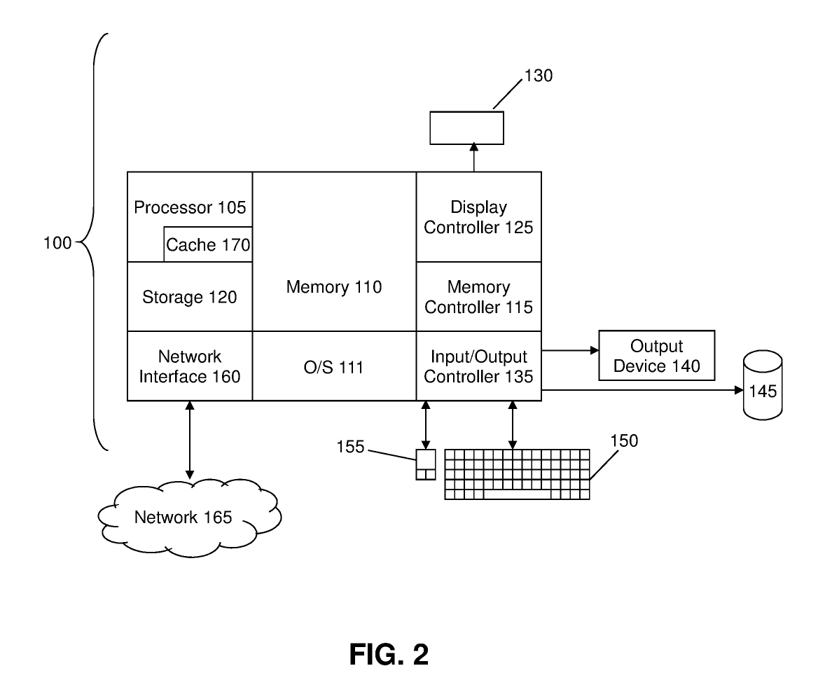

FIG. 2 illustrates a block diagram of a controller 100 for use in implementing a system or method according to some embodiments. The systems and methods described herein may be implemented in hardware, software (e.g., firmware), or a combination thereof. In some embodiments, the methods described may be implemented, at least in part, in hardware and may be part of the microprocessor of a special or general-purpose controller 38, such as a personal computer, workstation, minicomputer, or mainframe computer.

In some embodiments, as shown in FIG. 2, the controller 100 includes a processor 105, memory 110 coupled to a memory controller 115, and one or more input devices 145 and/or output devices 140, such as peripheral or control devices that are communicatively coupled via a local I/O controller 135. These devices 140 and 145 may include, for example, battery sensors, position sensors, cameras, microphones and the like. Input devices such as a conventional keyboard 150 and mouse 155 may be coupled to the I/O controller. The I/O controller 135 may be, for example, one or more buses or other wired or wireless connections, as are known in the art. The I/O controller 135 may have additional elements, which are omitted for simplicity, such as controllers, buffers (caches), drivers, repeaters, and receivers, to enable communications.

The I/O devices 140, 145 may further include devices that communicate both inputs and outputs, for instance disk and tape storage, a network interface card (MC) or modulator/demodulator (for accessing other files, devices, systems, or a network), a radio frequency (RF) or other transceiver, a telephonic interface, a bridge, a router, and the like.

The processor 105 is a hardware device for executing hardware instructions or software, particularly those stored in memory 110. The processor 105 may be a custom made or commercially available processor, a central processing unit (CPU), an auxiliary processor among several processors associated with the controller 38, a semiconductor based microprocessor (in the form of a microchip or chip set), a macroprocessor, or other device for executing instructions. The processor 105 includes a cache 170, which may include, but is not limited to, an instruction cache to speed up executable instruction fetch, a data cache to speed up data fetch and store, and a translation lookaside buffer (TLB) used to speed up virtual-to-physical address translation for both executable instructions and data. The cache 170 may be organized as a hierarchy of more cache levels (L1, L2, etc.).

The memory 110 may include one or combinations of volatile memory elements (e.g., random access memory, RAM, such as DRAM, SRAM, SDRAM, etc.) and nonvolatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 110 may incorporate electronic, magnetic, optical, or other types of storage media. Note that the memory 110 may have a distributed architecture, where various components are situated remote from one another but may be accessed by the processor 105.

The instructions in memory 110 may include one or more separate programs, each of which comprises an ordered listing of executable instructions for implementing logical functions. In the example of FIG. 2, the instructions in the memory 110 include a suitable operating system (OS) 111. The operating system 111 essentially may control the execution of other computer programs and provides scheduling, input-output control, file and data management, memory management, and communication control and related services.

Additional data, including, for example, instructions for the processor 105 or other retrievable information, may be stored in storage 120, which may be a storage device such as a hard disk drive or solid state drive. The stored instructions in memory 110 or in storage 120 may include those enabling the processor to execute one or more aspects of the systems and methods of this disclosure.

The controller 100 may further include a display controller 125 coupled to a user interface or display 130. In some embodiments, the display 130 may be an LCD screen. In some embodiments, the controller 100 may further include a network interface 160 for coupling to a network 165. The network 165 may be an IP-based network for communication between the controller 38 and an external server, client and the like via a broadband connection. The network 165 transmits and receives data between the controller 38 and external systems. In an embodiment, the external system may be the UAV 20, wherein the transmitting and receiving of data allows the controller 100 to determine when a condition (e.g. a route deviation, a player's vital sign parameters). In some embodiments, the network 165 may be a managed IP network administered by a service provider. The network 165 may be implemented in a wireless fashion, e.g., using wireless protocols and technologies, such as WiFi, WiMax, satellite, etc. The network 165 may also be a packet-switched network such as a local area network, wide area network, metropolitan area network, the Internet, or other similar type of network environment. The network 165 may be a fixed wireless network, a wireless local area network (LAN), a wireless wide area network (WAN) a personal area network (PAN), a virtual private network (VPN), intranet or other suitable network system and may include equipment for receiving and transmitting signals.

Systems and methods according to this disclosure may be embodied, in whole or in part, in computer program products or in controller 100, such as that illustrated in FIG. 2.

Monitoring a person or a group of people with a UAV 20 is a function that has many applications, such as, personal security, monitoring of a child going to school by parents, tracking the location of an elderly relative, and supervising the health status of players at a sporting event for example.

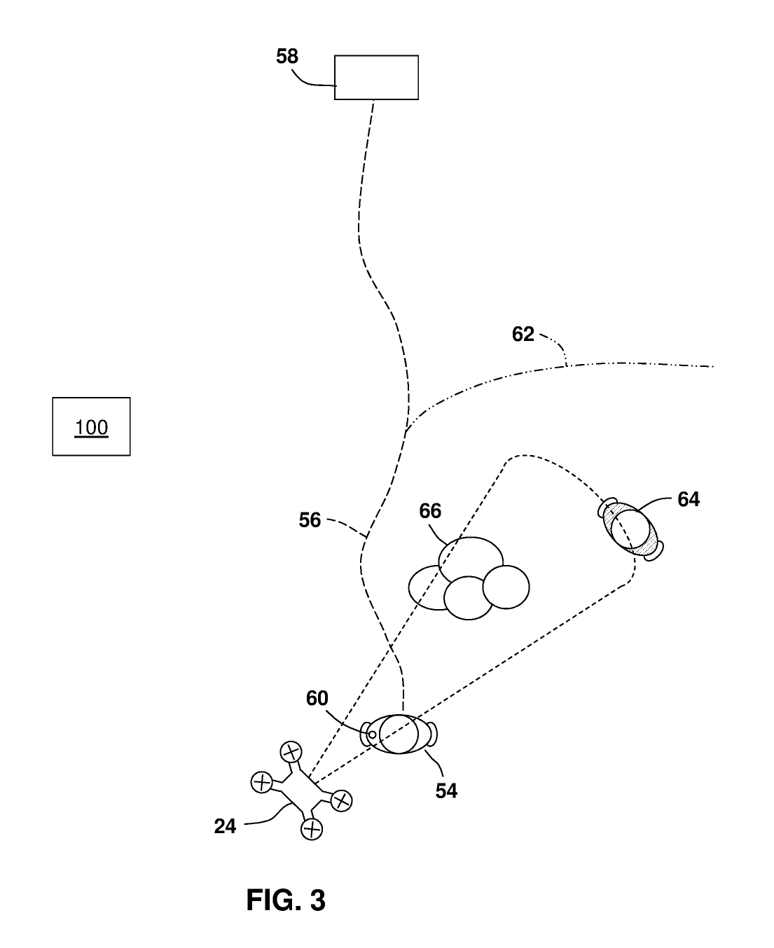

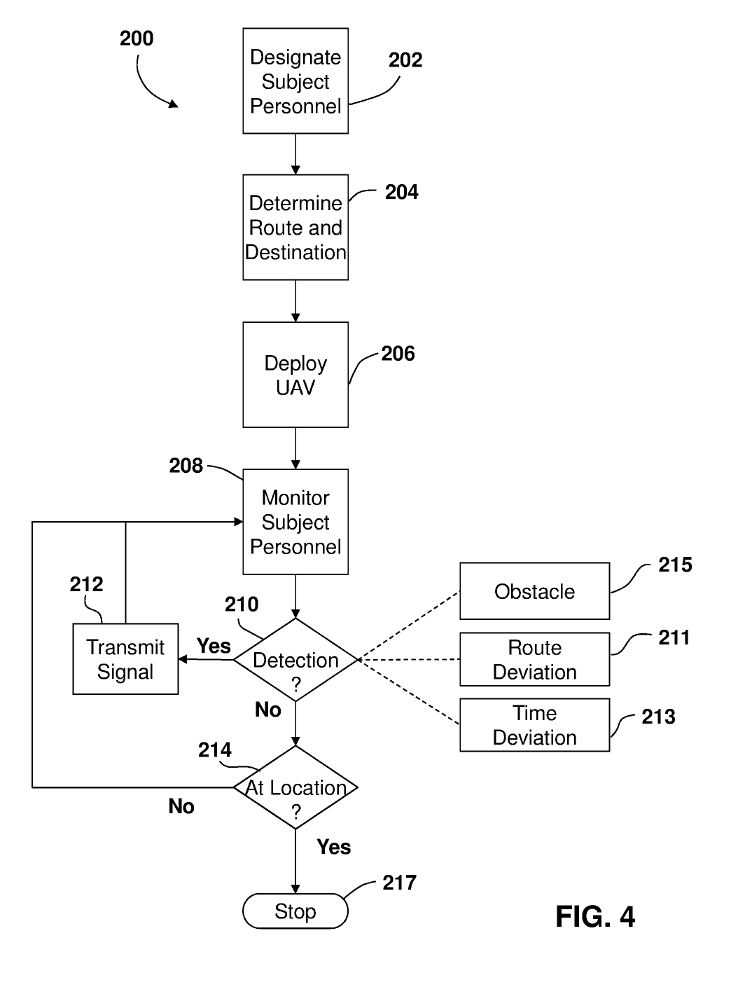

Referring now to FIG. 3 and FIG. 4, an embodiment is shown for monitoring with a UAV 20 the travel of a subject person 54 along a route 56 to an end destination 58. The UAV 20 may be in communication with the controller 100, such as via a wireless communications network for example. In an embodiment, the route 56 may be a predetermined route. In other embodiments, the route 56 may be a general area, meaning that the subject person 54 has options on the specific path traversed. In embodiments, the UAV 20 monitors the location of the subject person 54 with either a camera 48 or with a wireless transmitter 60 carried by the subject person 54. The positioning of the UAV 20, such as with GPS system 44 allows the controller 100 to determine if the subject person 54 is on the desired route 56 or if they have deviated along a different path 62 for example. In addition to monitoring the location of the subject person 54, the camera 48 may be used to monitor the presence of other persons 64 or obstacles 66 for example.

It should be appreciated that other sensors, such as the thermal imaging sensor 50 for example, may be used for monitoring the location of the subject person or for the presence of other persons 64. The thermal imaging sensor 50 may allow for monitoring the subject person (or other persons) when visual/optical sensors are less effective or ineffective, such as when the subject person is obscured by tree limbs or bushes. The heat signature measured by the thermal imaging senor 50 of the subject person allows the UAV 20 to determine the subject person's position. Further, other sensors, such as the acoustical sensor 52, may be used for monitoring the position of the subject person by monitoring sounds such as footsteps. The acoustical sensor 52 may also allow for the determination of a condition, such as if the subject person stumbles or falls, even when the subject person is not visible to optical type sensors.

Referring now to FIG. 4, a method 200 is provided for monitoring the subject person 54. The method 200 starts in block 202 where the subject person 54 or persons is designated. The designating of the person to be monitored may be performed such as by activating a wireless transmitter 60 that is carried by the subject person 54. The activating of the wireless transmitter 60 may be performed by the subject person 54, or by a third party (e.g. parents, school officials, public safety officers, etc.) The person may also be designated using image analysis techniques, wherein an image of the subject person 54 is provided to or acquired by the UAV 20. In an embodiment, the image of the subject person 54 may be transmitted to the UAV 20 by the subject person 54 or a third party (e.g. parents, schools officials, public safety officers, etc.) via controller 100 (FIG. 2). In still other embodiments, the designating of the person may be performed optically, such as by having the subject person carry a passive or active optical device, such as an light emitting diode or a reflective symbol for example, that is visible to a camera 48 or other optical sensor on the UAV 20. In an embodiment, the optical subject may emit or reflect light that is outside of the spectrum visible to a human eye.

It should be appreciated that while embodiments herein may describe a transmitter or optical subject as being carried by the subject person, this also includes the carrying or placing of the transmitter or optical subject on articles associated with the subject person 54. In some embodiments, the optical subject may be on a student's backpack for example.

The method 200 then proceeds to block 204 where the destination 58 and route 56 to be taken by the subject person 54 are determined. In some embodiments, a time parameter for the subject person to reach the destination location 58 or one or more intermediate locations may also be defined. In an embodiment, the route 56 is a specific path that the subject person 54 is to follow. In other embodiments, the route 56 is an area and the path followed by the subject person 54 may vary within the area. The defined area is the geographic region the person is allowed to move along. This allows the subject person some flexibility in how precisely they follow the path. In one embodiment, a threshold parameter may be defined for how far or long the subject person 54 may stray or deviate from the route 56.

The method proceeds to block 206 where the UAV 20 is deployed. In one embodiment, monitoring may be performed by a plurality of UAV's 20. In one embodiment where a plurality of UAV's 20 are used, the plurality of UAV's 20 may monitor the subject person 54 at the same time or serially. The method 200 then proceeds to block 208 where the subject person 54 is monitored as they move along the route 56. In an embodiment, the UAV 20 determines the position of the UAV 20 via GPS system 44. Using the personnel sensors 46, the location of the subject person 54 may be estimated, such as by determining a distance from the UAV 20 to the subject person and determining the three-dimensional distance from the UAV 20 to the subject person 54. The determination of the distance may be performed by one of the sensors on the UAV 20. The distance may be determined via triangulation using images acquired by the camera 48 for example. The distance may also be directly measured using a noncontact sensor such as a distance meter, a RADAR device, a LIDAR device or a SONAR device. Since the position of the UAV 20 is known via GPS system 44, the position of the subject person 54 may then be determined based on the GPS coordinates of the UAV 20 and the distance from the UAV 20 to the subject person. In an embodiment, the UAV 20 transmits the location information of the subject person 54 on a periodic or aperiodic basis to the controller 100.

The method 200 then proceeds to query block 210 where it is determined if a condition has occurred. In an embodiment, the condition may be a deviation 211 from the route 56, such as if the subject person proceeds along a path 62 instead of towards the destination location 58. In an embodiment, the condition may be a time deviation 213 from a defined time. For example, if the subject person 54 takes too long to proceed between intermediate points along the route or fails to process towards the destination location 58 within a predetermined amount of time. In still another embodiment, the condition could be an obstacle 215 or an unrecognized person is detected. In an embodiment, the undesirability parameters may be user-selected or defined. Such as a threshold for a deviation from the planned route 56 or the amount of time that has elapsed for example and/or a distance (ie, ½ block) deviation for a prescribed route.

When the query block 210 returns a positive, meaning a condition has been detected, the method 200 proceeds to block 212 where a signal is transmitted to the controller 100. The controller 100 may then perform one or more predetermined actions, such as alerting one or more persons (e.g. the subject person's parents or school officials) of the condition or the subject person's location for example. In one embodiment, the controller 100 transmits a signal (e.g. a cellular text message) to the subject person 54 notifying them of the condition. It should be appreciated that the condition may also be a medical condition and the transmitted signal may be sent to emergency personnel to assist the subject person 54. In some embodiments, the signal may be transmitted to a parent or guardian, a caretaker, a school employee or a public safety officer. The method 200 then loops back to block 208 and the UAV 20 continues to monitor the subject person 54.

When the query block 210 returns a negative, meaning no condition has been detected, the method 200 proceeds to block 214 where it is determined if the subject person 54 has reached the destination location 58. In one embodiment, the UAV 20 may use the personnel sensors 46, such as a camera 48 to acquire an image of the subject person 54 at the location. In this embodiment, the acquired image may be transmitted to the controller 100. When the query block 214 returns a negative, the method 200 loops back to block 208 and the UAV 20 continues to monitor the subject person 54. When the query block 214 returns a positive, meaning the subject person 54 is at the destination location 58, the method 200 proceeds to block 217 and stops.

It should be appreciated that in some embodiments, the subject person 54 may not be visible to optical sensors on the UAV 20 for a least a portion of the route 56. Such as when the subject person 54 may be riding in a vehicle for example. In these embodiments, the UAV 20 may utilize other means for monitoring the subject person 54, such as a radio frequency (RF) signal transmitted by a device carried by the subject person 54. As discussed above, the UAV 20 may also have sensors such as thermal imaging sensor 50 and acoustical sensor 52 that enable monitoring the subject person 54 when the subject person 54 is not visible to optical sensors. In another embodiment, the optical sensors on UAV 20 may determine the subject person 54 has entered a vehicle based at least in part on image analysis and automatically follows the vehicle until it is determined the subject person 54 has exited the vehicle.

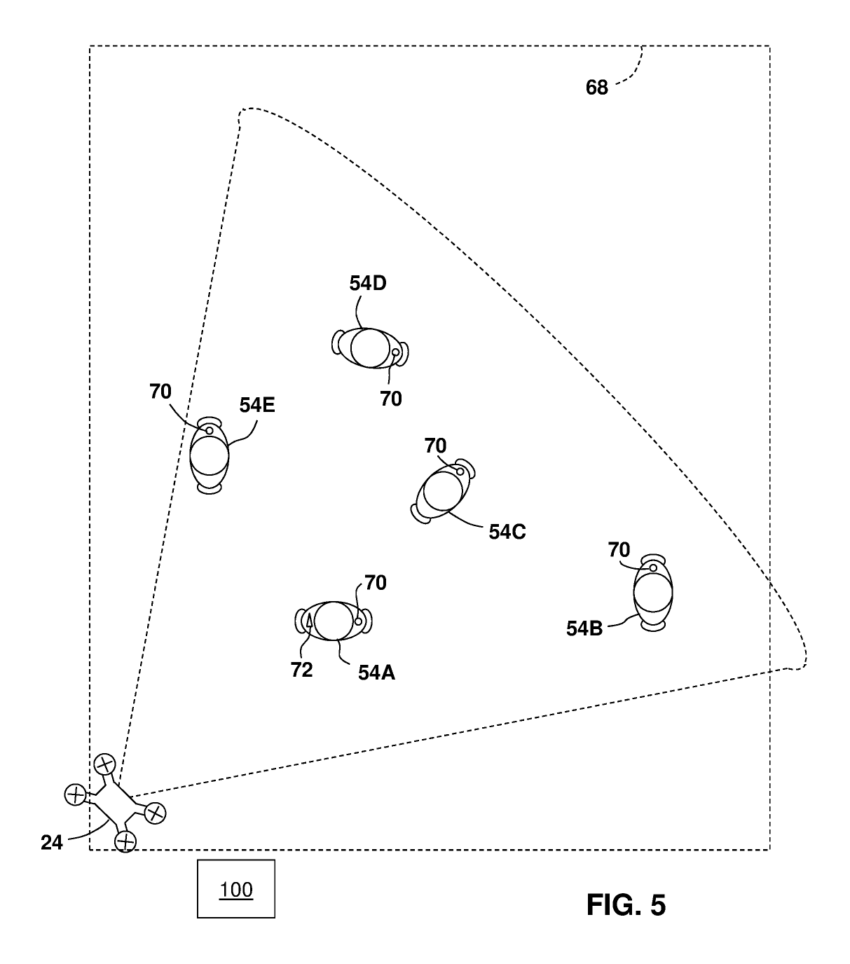

Referring now to FIG. 5 another embodiment is illustrated of a monitoring system for a group of people, such as players at an athletic contest. It should be appreciated that while embodiments herein describe players in an athletic contest, this is for exemplary purposes and the claimed invention should not be so limited. In other embodiments, the system may be arranged to monitor the status of other groups of individuals at a common location or event (e.g. people attending a concert). In this embodiment, one or more UAV's 20 are arranged to monitor a group of players 54A-54E. It should be appreciated that it is desirable to monitor attributes of athletes during an athletic contest to reduce the risk of a medical issue arising. Attributes that may be monitored include impacts/concussion-events and biometric parameters (e.g. blood pressure, heart rate, respiration rate, perspiration/moisture, temperature and blood oxygen levels) for example. In an embodiment, the athletic contest may take place on a field or court 68 and the UAV 20 may be configured to monitor the players 54A-54E within the boundaries of the field 68. As will be discussed in more detail below, to monitor these conditions the players 54A-54E may have one or more wearable devices that include sensors arranged to measure the biometric parameters. For example, the wearable device may include a blood pressure sensor, a heart rate sensor, a moisture sensor, a skin temperature sensor and a blood oxygen sensor (e.g., pulse oximetry). The monitored conditions of the players may be wirelessly communicated from the wearable device to the UAV 20 as discussed herein.

In the illustrated embodiments described herein, the storage and transmission of the biometric parameter data may be encrypted or otherwise secured to prevent access to the data by unauthorized persons.

It should be appreciated that while the illustrated embodiment shows a single UAV 20, this is for exemplary purposes and the claimed invention should not be so limited. In other embodiments, the multiple UAV's 20 may be used. For example, the players of each team may be monitored by separate UAV's 20 that only monitor one team. In another embodiment, each player 54A-54E may be monitored by an individual UAV 20.

In an embodiment, the players 54A-54E may have a communications device 70 that securely transmits information to the UAV 20. In an embodiment the communications device 70 may be a wireless transmitter that emits a radio frequency signal (e.g. Bluetooth or WiFi). In other embodiments, the communications device 70 may be an optical device, such as an LED light for example, that is positioned to be visible to the UAV 20. The communications device 70 may be coupled to a wearable device 72. The wearable device 72 may be comprised of one or more sensors or biotelemetry devices, such as but not limited to a hear rate monitor, a blood pressure monitor, a thermometer or temperature sensors, accelerometers, load cells, a respiratory rate monitor, a perspiration or moisture sensor and a blood oxygen sensor for example. In one embodiment, the communications device 70 cooperates with the wearable device 72 to transmit biometric vital information to the UAV 20. In another embodiment, the wearable device 72 may include a controller that compares the measurements from the sensors or biotelemetry devices to predetermined thresholds and determines when a biometric parameter is outside of a desired range. In one embodiment, the transmission of the out-of-range biometric parameter is transmitted optically via the communications device (e.g. a color coded signal) or via a radio-frequency signal. The UAV 20 may in turn transit the information to the controller 100. In one embodiment, the controller 100 may be a cellular device carried by coaching staff for the players 54A-54E.

In one embodiment, the UAV 20 may be used to determine if one or more players 54A-54D have received an impact that potentially could have caused a concussion. A concussion is a type of traumatic brain injury that may be caused during a sporting event due to an impact to the head. In one embodiment, the impact may be measured by accelerometers or load cells that are part of the wearable device 72. In another embodiment, the potential concussion event may be identified using a graphical or image analysis of images acquired by the UAV 20, such as via the camera 48. Using image analysis, movement and acceleration of the players head may be continuously or substantially continuously monitored during the athletic event. When the movement or acceleration of a players head, such as may be indicated by the movement of the head between two or more image frames from the camera for example, that indicates an impact on the head, a concussion-event may be detected. In an embodiment, when a potential concussion-event has been detected, the UAV 20 may perform actions to further confirm or gather additional information, such as but not limited to positioning the UAV 20 to acquire images of the player's eye with the camera 48. In some embodiments, the UAV 20 where the player is wearing a helmet, the UAV 20 may have to be positioned directly in front of the player. In other embodiments, the UAV 20 may be able to acquire the image of the player's eyes from a further distance using optical or digital zooming with the camera 48. From the acquired images, image analysis may be used to detect dilation of the pupil, or tracking the player involved in the potential concussion-event and identifying irregular walking patterns (e.g. staggering) by the player.

In an embodiment, the wearable-device 72 and the communications device 70 are not utilized and the UAV 20 monitors the players 54A-54D through sensors located on the UAV 20, such as a visual spectrum camera, an infra-red camera or a pyrometer for example. In this embodiment, the subject players may be identified by their location within a predefined area (e.g. the playing field or court) or by the color of their clothing/uniform. In this context, the area is a defined geographic region in which the person is monitored, such as the playing field, or the playing field and the areas adjacent the playing field.

The UAV 20 may communicate that a biometric parameter is outside of a desired predetermined range or the identification of a potential concussion-event to the controller 100. The predetermined range for each biometric parameter may be defined by the player, a coaches, the players parents or by authorized medical personnel. In one embodiment, the determination that a biometric parameter is outside of a desired range or a potential concussion-event has occurred may be performed by the UAV 20, the controller 100 or a combination of the foregoing. In an embodiment, the controller 100 may communicate with one or more remote computers (nodes) that may perform some or all of the diagnostic methods disclosed herein.

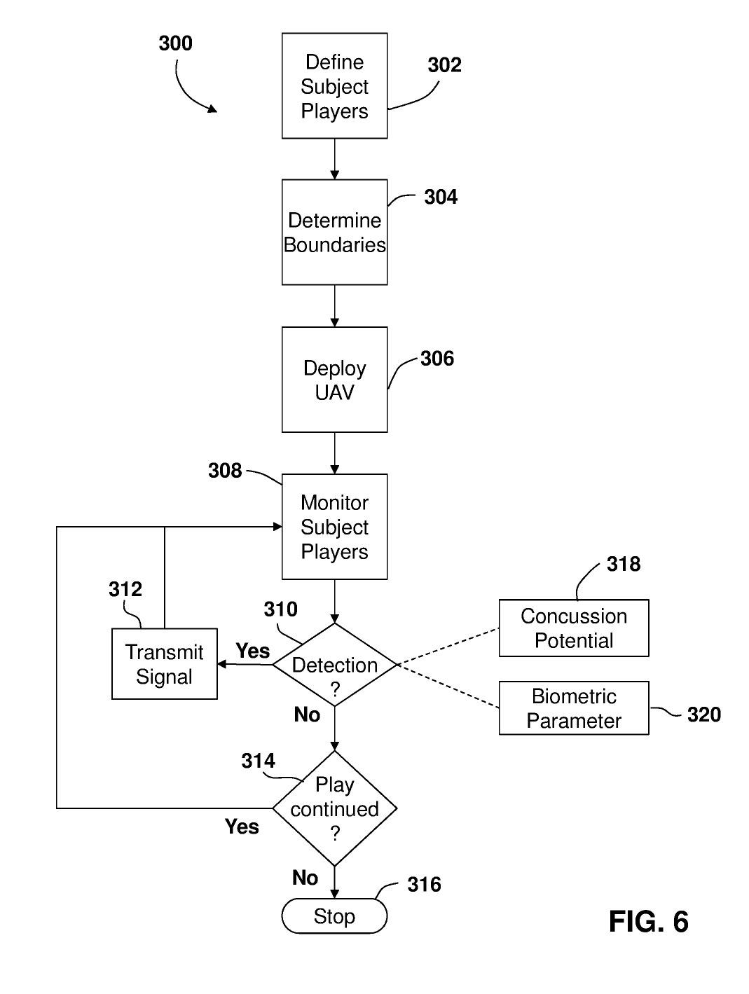

Referring now to FIG. 6, an embodiment is shown of a method 300 for monitoring a status of a group of people, such as the players 54A-54D. The method 300 begins in block 302 where the subject players to be monitored are defined. For example, the players on a particular team may be identified. The defining of the subject players may include the pairing of the communications devices 70 from the desired players to the UAV 20. The method 300 then proceeds to block 304 where the boundaries of the area to be monitored are defined. For example, it may be desirable to monitor the players 54A-54D when they are on the active portion of the playing field or court, but not when they are on the side-lines or otherwise out of bounds. The method 300 then proceeds to block 306 where the UAV 20 is deployed.

The subject players 54A-54D are monitored in block 308. Monitoring may include communications between the UAV 20 and the wearable-device 72 for example. Monitoring may further include image analysis of images acquired by a camera 48 on the UAV 20 for example. The method 300 then proceeds to query block 310 where a condition is detected. In an embodiment the detected condition may be the detection of a potential concussion-event 318 or a biometric parameter being outside of a desired range 320. In an embodiment, the sensors that measure the biometric parameters may include, but are not limited to, a hear rate monitor, a blood pressure monitor, a thermometer or temperature sensors, accelerometers, load cells, a respiratory rate monitor, a perspiration or moisture sensor and a blood oxygen sensor for example. The identification of a potential concussion-event may include image analysis of images acquired by a camera on the UAV 20, via sensors located on player, or a combination of the foregoing.

When the query block 310 returns a positive, meaning a condition is detected, the method 300 proceeds to block 312 and a signal is transmitted. In an embodiment, the signal may be an alert signal transmitted by the UAV 20 to the controller 100. The alert signal may include information on the identification of the player 54A-54D along with the detected condition or deviation of a biometric parameter relative to a threshold (e.g. heart rate crosses a threshold, lack of perspiration). It should be appreciated that depending on the biometric parameter, the crossing of a threshold may be due to the biometric parameter exceeding the threshold (e.g. heart rate above a threshold) or the biometric parameter falling below the threshold (e.g. the subject player stops perspiring and the level of skin moisture is less than a threshold). In an embodiment, the signal is transmitted to a portable computing device that is carried by a coach, training staff or medical personnel at the athletic event. The method 300 then loops back to block 308 and continues to monitor the players 54A-54D.

When the query block 310 returns a negative, meaning no condition has occurred, the method 300 proceeds to block 314 where it is determined if the athletic event is continuing (e.g. time has not expired). When query block 314 returns a positive, the method 300 loops back to block 308 and continues to monitor the players. When query block 314 returns a negative, the method 300 proceeds to block 316 and stops.

It should be appreciated that while the embodiment of FIG. 5 and FIG. 6 is directed to monitoring of a group of people with respect to a sporting event, this is for exemplary purposes and the claimed inventions should not be so limited. In other embodiments, groups of people may be monitored for conditions in other contexts.

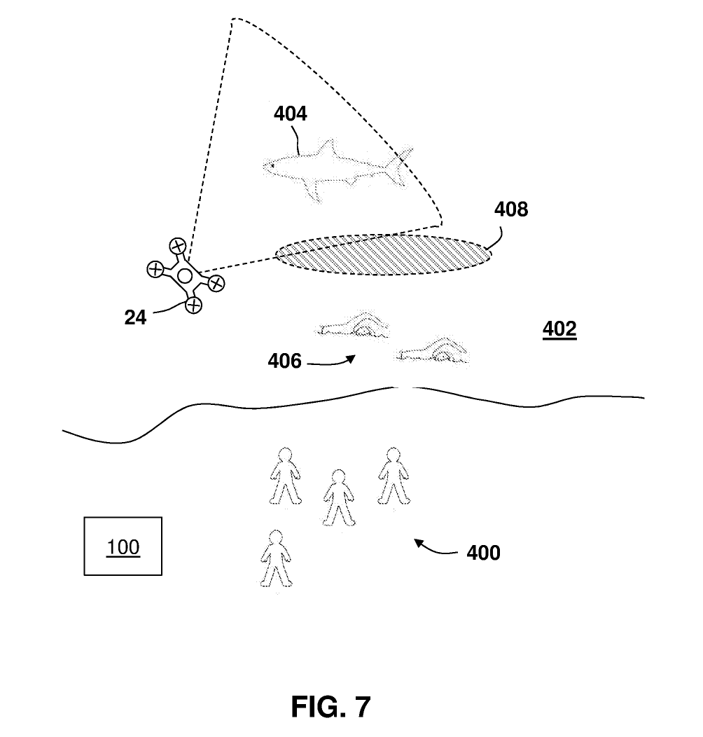

Referring now to FIG. 7 an embodiment is shown of a system for monitoring a group of people 400 in an area such as a beach. It should be appreciated that some wildlife, such as a shark at a beach, a bear at a park, or a lion at a nature reserve for example, may represent a condition may be monitored for and corrective action taken.

In the embodiment, of FIG. 4, the UAV 20 monitors the ocean 402 for the presence of a condition, such as a shark 404. This monitoring may be performed through the image analysis of images acquired by a camera on the UAV 20. It should be appreciated that while embodiments, herein may refer to the condition in the context of an animal, this is for exemplary purposes and the claimed invention should not be so limited. In other embodiments, the UAV 20 may use sensors to detect other conditions, such as a rip tide or waves of a predetermined size for example.

In an embodiment, upon detecting the condition the UAV 20 may transmit a signal to controller 100. In an embodiment, the group of people 400 may have access to an application on a mobile computing device that will alert them to the presence of the condition. In an embodiment, the mobile computing device may be a wearable device, such as a wrist band that provides haptic feedback to the wearer of the alert signal. This would allow the wearer to receive the alert signal even if they were swimming in the ocean 402.

In another embodiment, the UAV 20 may perform an action in response to the condition. In the embodiment where the condition is a shark 404, the action may be to deploy a means for diverting the shark 404 away from swimmers 406. Such an action may include deploying a chemical shark repellent (e.g. sodium lauryl sulfate) in an area 408 between the shark 404 and the swimmers 406. Other actions may include producing an acoustical signal or an electrical impulse to repel the shark 404. In still other embodiments, the UAV 20 may provide audio or visual alerts to the people 400 of the presence or location of the shark 404.

It should be appreciated that while embodiments herein refer to a controller 100 as controlling and managing the UAVs, this is for exemplary purposes and the claims should not be so limited. In other embodiments, the controlling and managing of the UAVs may be performed by a plurality of controllers, a distributed computing environment or a cloud computing environment. It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

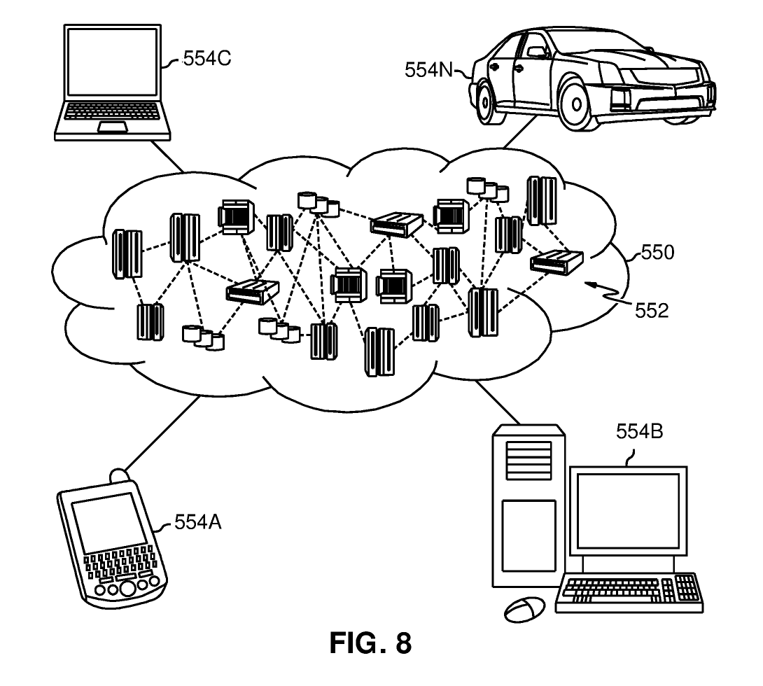

Referring now to FIG. 8, illustrative cloud computing environment 550 is depicted. As shown, cloud computing environment 350 comprises one or more cloud computing nodes 552 with which local computing devices used by cloud consumers, such as, for example, personal digital assistant (PDA) or cellular telephone 554A, desktop computer 554B, laptop computer 554C, and/or automobile computer system 554N may communicate. Nodes 552 may communicate with one another. They may be grouped (not shown) physically or virtually, in one or more networks, such as Private, Community, Public, or Hybrid clouds as described hereinabove, or a combination thereof. This allows cloud computing environment 550 to offer infrastructure, platforms and/or software as services for which a cloud consumer does not need to maintain resources on a local computing device. It is understood that the types of computing devices 554A-N shown in FIG. 12 are intended to be illustrative only and that computing nodes 552 and cloud computing environment 550 can communicate with any type of computerized device over any type of network and/or network addressable connection (e.g., using a web browser).

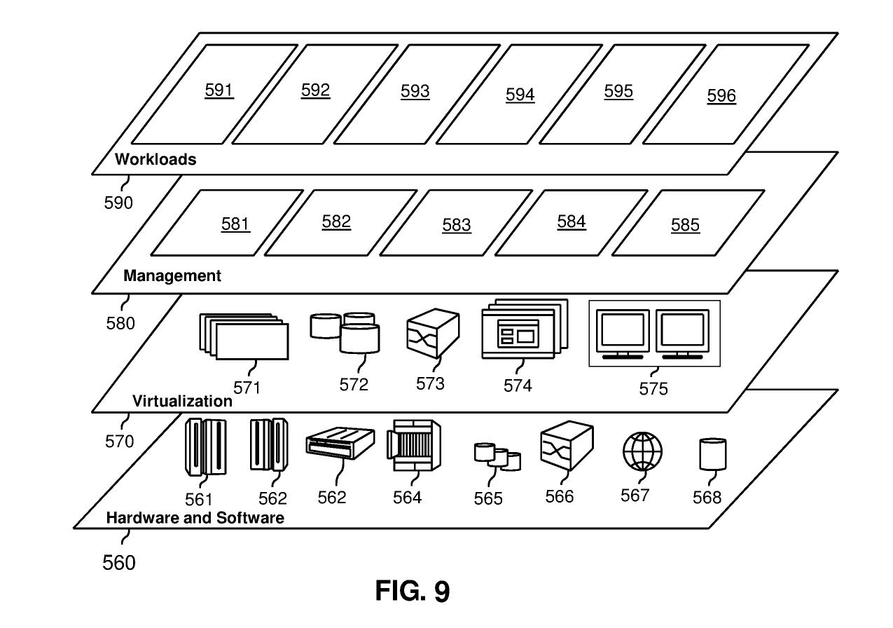

Referring now to FIG. 9, a set of functional abstraction layers provided by cloud computing environment 550 (FIG. 8) is shown. It should be understood in advance that the components, layers, and functions shown in FIG. 9 are intended to be illustrative only and embodiments of the invention are not limited thereto. As depicted, the following layers and corresponding functions are provided:

Hardware and software layer 560 includes hardware and software components. Examples of hardware components include: mainframes 561; RISC (Reduced Instruction Set Computer) architecture based servers 562; servers 563; blade servers 564; storage devices 565; and networks and networking components 566. In some embodiments, software components include network application server software 567 and database software 568.

Virtualization layer 570 provides an abstraction layer from which the following examples of virtual entities may be provided: virtual servers 571; virtual storage 572; virtual networks 573, including virtual private networks; virtual applications and operating systems 574; and virtual clients 575.

In one example, management layer 580 may provide the functions described below. Resource provisioning 581 provides dynamic procurement of computing resources and other resources that are utilized to perform tasks within the cloud computing environment. Metering and Pricing 582 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 583 provides access to the cloud computing environment for consumers and system administrators. Service level management 584 provides cloud computing resource allocation and management such that required service levels are met. Service Level Agreement (SLA) planning and fulfillment 585 provides pre-arrangement for, and procurement of, cloud computing resources for which a future requirement is anticipated in accordance with an SLA.

Workloads layer 590 provides examples of functionality for which the cloud computing environment may be utilized. Examples of workloads and functions which may be provided from this layer include: mapping and navigation 591; software development and lifecycle management 592; virtual classroom education delivery 593; data analytics processing 594; transaction processing 595; and a UAV positioning and monitoring management 596. The UAV positioning and monitoring management 596 may perform one or more methods that allow monitoring of a person or persons, such as but not limited to the methods described in reference to FIG. 4 and FIG. 6 for example.

Technical effects and benefits of some embodiments include monitoring a person or persons for a condition within defined area using an autonomously operated aerial vehicle.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
defining a subject to be monitored for health and safety conditions with an unmanned aerial vehicle (UAV), the UAV having at least one sensor;
defining an area associated with the subject;
defining a condition associated with the subject;
deploying the UAV to monitor the subject;
detecting the condition within the area; and
transmitting a signal based on the detecting of the condition.

2. The method of claim 1, further comprising defining a route and a destination for the subject.

3. The method of claim 2, wherein the monitoring of the subject includes determining a location of the subject.

4. The method of claim 3, wherein the detecting of the condition includes detecting a route deviation, a time deviation or an obstacle.

5. The method of claim 1, wherein the defining the subject includes defining a plurality of subjects.

6. The method of claim 5, wherein the detecting the condition includes detecting a potential concussion event.

7. The method of claim 6, wherein the at least one sensor includes a camera and the detecting of the potential concussion event comprises analyzing at least one image acquired by the camera.

8. The method of claim 6, wherein the detecting of the condition includes determining a biometric parameter associated with one of the plurality of subjects that crosses a threshold.

9. The method of claim 8, further comprising at least one biometric sensor operably coupled to each of the plurality of subjects, wherein the at least one biometric sensor is selected from a group comprising a heart rate monitor, a blood pressure monitor, a thermometer or temperature sensors, accelerometers, load cells, a respiratory rate monitor, a perspiration or moisture sensor and a blood oxygen sensor.

10. The method of claim 1, further comprising performing an action with the UAV when the condition is associated with an animal.

11. A system comprising:
a UAV having at least one sensor;
a memory having computer readable instructions; and
one or more processors for executing the computer readable instructions, the computer readable instructions comprising:
defining a subject to be monitored for health and safety conditions with the UAV;
defining an area associated with the subject;
defining a condition associated with the subject;
deploying the UAV to monitor the subject;
detecting the condition within the area; and
transmitting a signal based on the detecting of the condition.

12. The system of claim 11, wherein:
the computer readable instructions further comprise defining a route and a destination for the person;
the monitoring of the subject includes determining a location of the subject; and
the detecting of the condition includes detecting a route deviation, a time deviation or an obstacle.

13. The system of claim 11, wherein the defining the person includes defining a plurality of subjects.

14. The system of claim 13, wherein the detecting the condition includes detecting a potential concussion event or determining a biometric parameter associated with one of the plurality of subjects that crosses a threshold.

15. The system of claim 14, wherein the at least one sensor includes a camera and the detecting of the potential concussion event includes an image analysis of images acquired by the camera.

16. The system of claim 11, further comprising at least one biometric sensor operably coupled to the subject, wherein the at least one biometric sensor is selected from a group comprising a hear rate monitor, a blood pressure monitor, a thermometer or temperature sensors, accelerometers, load cells, a respiratory rate monitor, a perspiration or moisture sensor and a blood oxygen sensor.

17. A computer program product for a monitoring a subject for a health and safety condition, the computer program product comprising a computer readable storage medium having program instructions embodied therewith, the program instructions executable by a processor to cause the processor to perform:
defining the subject to be monitored with an unmanned aerial vehicle (UAV), the UAV having at least one sensor;
defining an area associated with the subject;
defining the condition associated with the subject;
deploying the UAV to monitor the subject;
detecting the condition within the area; and
transmitting a signal based on the detecting of the condition.

18. The computer program product of claim 17, wherein the program instructions further comprising defining a route and a destination for the subject, wherein the monitoring of the subject includes determining a location of the subject and the detecting of the condition includes detecting a route deviation, a time deviation or an obstacle.

19. The computer program product of claim 17, wherein the defining the subject includes defining a plurality of subjects, and the detecting the condition includes detecting a potential concussion event or a biometric parameter associated with one of the plurality of subjects that crosses a threshold.

20. The computer program product of claim 17, wherein the program instructions further comprise performing an action with the UAV when the condition is associated with an animal.
